(19) United States
(12) Patent Application Publication
(10) Pub. No.: US 2010/0029542 A1
(43) Pub. Date: Feb. 4, 2010

(54) PROTEIN STERILISATION BY RADIATION AND ADDITION OF A STABILISING COMPOSITION

(76) Inventor: Jan Jezek, Bedfordshire (GB)

Correspondence Address: ELMORE PATENT LAW GROUP, PC, 515 Groton Road, Unit 1R, Westford, MA, 01886, US

(21) Appl. No.: 12/491,971

(22) Filed: Jun. 25, 2009

Related U.S. Application Data
(63) Continuation of application No. PCT/GB2007/004966, filed on Dec. 21, 2007.

(30) Foreign Application Priority Data
Dec. 29, 2006 (GB) 0626021.0

(51) Int. Cl. A61K 38/02; B01J 19/08

(52) U.S. Cl. 514/2; 204/157.64

(57) ABSTRACT

A method of sterilising a protein, comprises exposing to ionising radiation an at least substantially dry composition comprises a protein and a protective compound or combination of protective compounds having both of the following characteristics: (i) a rate of reaction with singlet oxygen greater than 1×10 7 L mol−1 S−1; (ii) being a reducing agent whilst at the same time containing a proton dissociable group with a pKa no more than 3 units from the pH of the composition. The compound having characteristic (i) is selected from histidine, thiamine and tryptophan, the compound having characteristic (ii) is selected from methionine, malate, citrate, lactate and tiron. The radiation is gamma radiation or electron beam, whereby the preferred dose is 15-40 kGy.

### RELATED APPLICATION

This application is a continuation of International Application No. PCT/GB2007/004966, which designated the United States and was filed on Dec. 21, 2007, which claims priority under 35 U.S.C. §119 or 365 to United Kingdom Application No. 0626021.0, filed on Dec. 29, 2006. The entire teachings of the above applications are incorporated herein by reference.

### FIELD OF THE INVENTION

This invention relates to the stabilisation of proteins, particularly of proteins in a solid state, for example in a non-liquid state where water is removed partially or fully from an aqueous solution by drying or by freeze-drying. More specifically, the invention relates to the stability of proteins in the presence of ionising radiation, particularly at ambient temperature or slightly above.

### BACKGROUND OF THE INVENTION

Many proteins are unstable and are susceptible to degradation and consequent loss of activity under certain conditions. Particular difficulties arise where the protein is required to be in a sterile condition.

One effective sterilisation technique involves exposure to ionizing radiation, e.g. gamma radiation or electron beam radiation. Sterilisation by exposure to ionising radiation is a particularly aggressive process, typically requiring doses of 25 to 40 kGy. These conditions are damaging to proteins, particularly in a liquid state due to the generation of free radicals by radiolysis of water (predominantly hydroxyl radical and hydrated electron) that, in turn, attack vulnerable groups at the protein surface.

Gamma radiation is one of several types of high-energy ionizing radiation. It consists of high energy photons that are emitted by nuclei of radioactive atoms (e.g. cobalt 60). The chemical and biological effects of ionizing radiation originate from two basic types of interactions. For direct action, the radiation energy is deposited directly in target molecules. For indirect action, the initial absorption of energy is by the external medium, leading to the production of diffusive intermediates which then attack the targets.

It is predominantly the indirect action that causes damage to chemical species dissolved in water. This means that the radiation first interacts with the solvent (i.e. water) to give rise to various reactive species, by the process of radiolysis. These reactive species then react with other solutes present in the solution (e.g. proteins). Thus, in order to protect the dissolved species against the effects of gamma rays, it is necessary to mitigate the adverse effects of the reactive species generated by radiolysis of water.

The precise mechanism of the ionising radiation in the non-aqueous dry state is considerably less clear. Although the direct action may be of some importance, it is believed that the indirect action contributes significantly to the damage caused by ionising radiation on chemical species in the dry state. This means that the radiation first interacts with molecules of surrounding air to give rise to various reactive species, either in the gaseous state or dissolved in the residual water. These reactive species react subsequently with the chemical species present in the irradiated sample (e.g. proteins).

Of the major components of air, it is particularly oxygen that is prone to radiolysis, generating ions, excited atoms and molecules, and free radicals that react readily with other chemical species. The radiolysis of molecular oxygen has been of continuing interest because of the importance of these reactions in the atmosphere. Four primary reactions have been identified:

O₂→O₂⁺e⁻

O₂→O⁺+O.+e⁻

O₂→2 O.

O₂→*O₂

The species generated by the primary reactions react further (where M is another molecule of oxygen or a solid surface to remove excess energy) as follows:

O₂⁺+e⁻→2 O.

O⁺+e⁻→O.

O₂+e⁻→O₂⁻

O₂+O.+M→O₃+M

As shown above, irradiation of oxygen by ionising radiation triggers a complex series of reactions leading to the following main products:
*O₂, i.e. a short-lived, excited state of oxygen; typically singlet oxygen (¹O₂).
Superoxide anion radical (O₂⁻)
Oxygen atom (O.)
Oxygen molecule cation (O₂⁺) and oxygen atom cation (O⁺)
Ozone (O₃)

In general, removal of water from the protein sample improves the stability of proteins in the presence of ionising radiation. This is proposed in US2003/0012687 as a means of improving the recovery of protein activity and structure after gamma irradiation. It is also proposed that replacement of water by an alternative solvent, such as ethanol or acetone, can improve a protein's stability when subjected to ionising radiation. A number of examples demonstrate the effect of water removal on protein stability during gamma irradiation.

Many therapeutic proteins are rendered in the dry form by drying or freeze-drying. These products can be sterilised conveniently by ionizing radiation. Typically, greater than 95% recovery of both functional activity and structural integrity of the protein following sterilisation by ionising radiation will be required. Formulations resulting in lower recovery following exposure to ionizing radiation are very unlikely to be considered for therapeutic applications. In most cases, the low water content alone does not guarantee the required recovery of functional activity and structural integrity of the protein following sterilisation by gamma radiation, so other measures must therefore be taken to ensure sufficient stability of the protein.

Another measure that can be considered for maximising the recovery of protein activity and structure is that of reducing the temperature of the sample whilst it is undergoing irradiation. This is proposed in US2003/0012687. In most of the examples, protein samples were irradiated by gamma rays at 4° C. or below. However, this is impractical on a large scale. Large-scale industrial sterilisation by gamma or e-beam irradiation is routinely carried out at ambient temperature. In fact, it is known to those skilled in the art that, if no cooling is employed, the temperature of samples during exposure to gamma or e-beam radiation rises above ambient. For these reasons, most examples shown in US2003/0012687 are of research interest only.

Yet another measure that can be considered in order to achieve greater than 95% retention of structural and functional characteristics of a protein after gamma or e-beam irradiation is addition of excipients into the protein formulation. A number of excipients are suggested in US2003/0012687 that can improve the protein recovery either alone or typically in combination with other measures such as reducing the temperature. The efficiency of a small number of excipients in improving the recovery of proteins in dry state after gamma irradiation is demonstrated in several examples and some generalisations are made. The excipients are defined generally under the terms “antioxidants” and “free radical scavengers” which encompass a great number of compounds. No more precise definitions or specifications of these terms are disclosed.

The term “free radical scavenger” refers typically to a compound that can react very readily with any one free radical. There are a great number of unstable chemical species with one or more unpaired electrons that can be referred to as free radicals. Most compounds are known to react with free radicals. The compounds that react with the highest rate, which are therefore most effective in sequestering the free radicals, are called “free radical scavengers”. However, the rate of reaction of a given compound with different free radicals varies considerably. Consequently, a given compound can be referred to as an effective scavenger of one free radical, but can be completely ineffective in scavenging another free radical. For example, the malate anion is known to be a very effective scavenger of superoxide. However, the reaction rate of the malate anion with another free radical called the hydrated electron is more than three orders of magnitude lower than that of many other compounds. Similarly, citrate is known to be an effective scavenger of superoxide but not of singlet oxygen, nor of hydrated electrons, nor of hydroxyl radicals. Adenosine is a very effective scavenger of both hydrated electrons and hydroxyl radicals, but not of singlet oxygen. The enzyme superoxide dismutase is only effective in scavenging superoxide, but has no effect on the activity of other free radicals. These are only a few examples of compounds whose efficiency of scavenging free radicals is very selective to particular free radical species.

So, whilst the term “free radical scavenger” gives some indication of the properties of a compound thus described, further definition is needed to clarify the actual reactivity of the compound with individual free radicals.

There are many definitions of the term antioxidant. In the broadest sense, an antioxidant is a substance that when present in low concentrations relative to an oxidisable substrate significantly delays or reduces oxidation of the substrate. Typically, however, the term relates only to substances of physiological importance, i.e. either those that play a role in human or animal metabolism or those found in human or animal diet. They also typically relate to counter-acting oxidative effects caused by various free radicals, so the definition of an antioxidant is sometimes presented as identical to that of a “free radical scavenger”. However, this is not always the case, as some free radicals do not exert their reactivity through oxidation. For example, the free radical hydrated electron is a very strong reducing agent completely incapable of any oxidative damage.

The examples in US2003/0012687 are of varying combinations of compounds that show improvement of stability of model proteins in the dry state (typically freeze-dried) through gamma irradiation. Typically, these are combinations of ascorbate, glycylglycine, urate and trolox. In addition, lipoic acid, glutathione, cysteine and several flavinoids such as epicatechin or rutin are also shown to have some protective effect. Most of these experiments were carried out at 4° C. or below, to maximise the recovery of the protein activity or structural integrity following irradiation. In some cases, the combination of excipients (mostly ascorbate and glycylglycine), together with reduced temperature, led to greater than 95% recovery of protein activity following irradiation. Nevertheless, this was only the case if the protein, such as a monoclonal antibody, inherently manifested good recovery (typically 60-70%) following gamma irradiation in the absence of the excipients. In our experimental experience, such good stability of unprotected protein is rather rare. No example in US2003/0012687 demonstrates >95% recovery of protein activity in dry state following exposure to ionising radiation at ambient temperature. Furthermore, some of the excipients used in the examples of US2003/0012687 would not be considered for use in therapeutic formulations due to their cost (e.g. epicatechin) or their safety (e.g. urate, rutin).

Post-sterilisation recovery efficiency is particularly important for therapeutic proteins. Known methods and materials do not provide reliable means for achieving recoveries of greater than 95% activity or structural integrity after application of ionising radiation at the industry standard dose level (25-40 kGy). Such recovery efficiency is only rarely reported, and, in those cases where the recovery is sufficient, the protein concerned is always one that has a high intrinsic resistance to ionising radiation, such as certain monoclonal antibodies. Yet, for any therapeutic application, recoveries of less than 95% would be unacceptable. Thus, there exists a need for technology that will reliably provide more than 95% recovery of the protein, after exposure to fulldose ionising irradiation.

The selection of suitable stabilising agents is also very important. As discussed above, the prior art identifies very broad classes or types of compound (e.g. “free radical scavenger” or “anti-oxidant”) as potential stabilizing agents. The immense number of compounds that fit within these general classes makes the job of selecting suitable protective agents (excipients) difficult. An individual skilled in the art and knowledgeable about such aspects of chemistry would be confronted with the need to screen many thousands of compounds, especially since the available specific examples do not provide adequate performance. The vast majority of these compounds turn out to be ineffective. No clear teaching exists by which an individual ordinarily skilled in the art can simply and reliably identify those rare, medically acceptable protein stabilising agents that will provide >95% recovery through gamma irradiation of dry protein formulations. Thus, there is a need for new understanding and clear teaching on what chemical features are needed to provide the required protection, so that effective excipients can be identified and formulated efficiently and accurately.

### SUMMARY OF THE INVENTION

It has surprisingly been found that many compounds that fit the generally accepted definitions of antioxidants and/or of free radical scavengers, either alone or in combination, cause inadequate improvement of stability of model proteins whilst irradiated by ionising radiation. Many combinations of antioxidants and other “free radical scavengers” are capable of causing good improvement in stability of the dry proteins during gamma sterilisation, but it has been found that it is only very specific combinations of excipients that are capable of conferring protection of protein while sterilised by ionising radiation at ambient temperature by an industry-standard sterilising service that would be sufficient for a therapeutic formulation of the protein.

In one aspect, the invention provides a method of sterilising a protein in a dry state, comprising bringing the protein into contact with a protective compound or combination of protective compounds having both of the following characteristics:

(i) a good rate of reaction (i.e. rate constant k>1×10⁷ L mol⁻¹ s⁻¹ at ambient temperature) with singlet oxygen; and

(ii) a scavenger of superoxide anion effective in dry state, i.e. a reducing agent, preferably a mild reducing agent (with E⁰ no less than +0.1 V), which at the same time is capable of exchanging a proton readily with the superoxide radical; and

exposing the protein and protective compound(s) to ionising radiation.

Optionally, the composition contains an additional reducing agent, preferably a mild reducing agent (with E⁰ no less than +0.1 V).

The protection may be complete, i.e. with 100% retention of activity, so that no activity is lost on exposure to ionising radiation, or may be partial, with less than 100% retention of activity, so that some (but not all) activity is lost on exposure to ionising radiation. The retention of activity is preferably at least 50%, more preferably at least 60%, 70%, 80% or 90%, most preferably at least 95%.

The ionising radiation is typically in the form of gamma radiation, electron beam radiation or X-ray radiation.

The invention also provides a composition comprising a protein in a dry state and a protective compound or combination of protective compounds having the following characteristics:

(i) a good rate of reaction (i.e. rate constant k>1×10⁷ L mol⁻¹ s⁻¹ at ambient temperature) with singlet oxygen

(ii) a scavenger of superoxide anion effective in dry state, i.e. a reducing agent, preferably a mild reducing agent (with E⁰ no less than +0.1 V), which at the same time is capable of exchanging a proton readily with the superoxide radical.

Optionally, the composition contains an additional reducing agent, preferably a mild reducing agent (with E⁰ no less than +0.1 V). The composition has desirably been sterilised by exposure to ionising radiation. The invention covers a protein in microbiologically sterile condition, after exposure to ionizing radiation.

In all aspects of the invention, the pH of the composition which contains the protein and the protective compound(s) may be adjusted to a required value, for example a value that ensures best heat stability of the protein during sterilisation and subsequent to the sterilisation. Typically, proteins will be formulated at a pH between 4 to 9. Most therapeutic proteins or proteins used for diagnostic purposes will be formulated at pH 5 to 8, typically at pH 5 to 7, most typically at pH around 6.

Small peptides comprising fewer than 20 amino acids which contain at least one disulphide bridge are likely to require formulating at pH between 4 to 6, typically around 5 to ensure optimum stability. This is because the stability of the disulphide bond is best at pH between 4 to 5. Therefore, in a further aspect, the invention also provides a composition comprising a peptide having fewer than 20 amino acids in a dry state and a protective compound or combination of protective compounds having the following characteristics:

(i) a good rate of reaction (i.e. rate constant k>1×10⁷ L mol⁻¹ s⁻¹ at ambient temperature) with singlet oxygen; and

(ii) a scavenger of superoxide anion effective in dry state, i.e. a reducing agent, preferably a mild reducing agent (with E⁰ no less than +0.1 V), which at the same time is capable of exchanging a proton readily with the superoxide radical;

wherein the pH of the composition is about 5.

Optionally, the composition contains an additional reducing agent, preferably a mild reducing agent (with E⁰ no less than +0.1 V). The composition has desirably been sterilised by exposure to ionising radiation.

### DETAILED DESCRIPTION OF THE INVENTION

The present invention arose from an analysis of the effects of ionizing radiation on proteins in the absence of water and the subsequent development of a model that enables selection of a compound or, more typically, a combination of compounds capable of protecting a protein in a solid state against the detrimental effects of ionising radiation to achieve recovery of functional activity and structural integrity that would be acceptable for therapeutic applications.

Since commercial production of sterile solid-state formulations of therapeutic proteins is one of the main applications of the present invention, there is an emphasis on inexpensive excipients listed as GRAS and, preferably, listed as inactive ingredients in FDA-approved therapeutic products. Industry standard gamma radiation (25-40 kGy) at ambient temperature may be used as a model ionising radiation. Degradation of proteins in dry formulations caused by the indirect action of ionising radiation is mediated by reactive oxygen species in gaseous state or dissolved in the residual water. Degradation of biological systems by reaction with reactive oxygen species and other free radicals is well known, and has been associated with many forms of tissue damage, disease and with the process of aging. Such interactions are normally considered in aqueous solutions, an environment typical for most biological systems. Consequently, the scientific literature is rife with information on free-radical mediated degradation of various biological and biochemical systems in aqueous solutions. Such information on reactions of reactive oxygen species in dry compositions is scarce. Nevertheless, reactive oxygen species are known to be produced in the gaseous state, so reactions with chemical species at the solid-gas interface can be expected to occur readily. Furthermore, traces of residual water facilitating the effects of the reactive species in the dissolved state can be expected, even in very dry samples.

A composition of the invention typically contains no more than 10%, preferably no more than 5, 4, 3, 2, 1 or 0.5%, water by weight.

Due to their considerable reactivity, the reactive oxygen species are believed to be the source of indirect radiation damage in dry protein samples even if the samples are irradiated in an oxygen-free atmosphere (e.g. if the sample is kept under nitrogen). This can be explained by the fact that, in the nitrogen atmosphere, some oxygen will stay adsorbed at the protein surface owing to its hydrophobicity. Strong hydrophobic interactions are possible between oxygen molecules and hydrophobic parts of the protein. Consequently, whilst the stability of proteins can be improved markedly when sterilised by ionising radiation if the proteins are placed under nitrogen, some protection against the oxygen reactive species is still necessary.

Protection from damage caused by the reactive oxygen species can be achieved through sacrificial molecules that react with, and thereby “scavenge”, the reactive species. So, in order to confer protection of a dry composition of a protein subjected to ionising radiation, it is necessary to add one or more compounds that react readily with one or more products of radiolysis of gaseous oxygen. In order to achieve very high recovery of the protein activity and structural integrity following sterilisation by ionising radiation, it is essential to add compounds that can scavenge effectively all of the major reactive chemical species generated by radiolysis of oxygen.

The ability of a compound to act as “scavenger” of a given reactive oxygen species depends on its readiness to react with the species. This can be expressed quantitatively using a rate constant of the reaction between the reactive chemical species and the scavenging species. The rate constants for the reactions of a large selection of compounds with singlet oxygen, including details of experimental methods used, can be obtained from a website maintained by the Radiation Chemistry Data Center (RCDC) of the Notre Dame Radiation Laboratory (University of Notre Dame, IN, USA). This is an information resource dedicated to the collection, evaluation, and dissemination of data characterising the reactions of transient intermediates produced by radiation, chemical and photochemical methods, reached through the following link: http://www.rcdc.nd.edu/compiiations/SingOx/TOC.HTM. The rate constants, including details of the experimental methods, can also be found in the following publication: Wilkinson F., Helman W. P., Ross A. B.: Rate Constants for the Decay and Reactions of the Lowest Electronically Excited Single State of Molecular Oxygen in Solution. An Expanded and Revised Compilation. J. Phys. Chem. Ref. Data 24: 663-1021 (1995). The contents of this and other references identified herein are incorporated by reference.

Although these rate constant values were measured when the selected chemical species were dissolved in specified solvents, it can be assumed that they reflect their reactivity in a dry state. This is especially relevant, since traces of solvents (typically free or bound water) can be expected in virtually any dry sample of a protein. A reaction rate threshold of 10⁷ L mol⁻¹ s⁻¹ was chosen (on the basis of an informed judgment) to select the effective scavengers of singlet oxygen.

Apart from scavengers of singlet oxygen, there is a small number of compounds that can eliminate singlet oxygen reactivity without engaging in chemical reactions. These compounds are known as singlet oxygen quenchers. Typical examples of singlet oxygen quenchers are 1,4-diazabicyclooctane, α-tocopherol, and β-carotene (Halliwell, 1999).

If no quantitative kinetic data are available then a qualitative approach can be applied to selection of scavengers of a given free radical. This means that a chemical species is considered to be an effective scavenger of a given free radical if such a qualitative description can be found in the scientific literature. Such qualitative descriptions can readily be found of scavengers of singlet oxygen, superoxide and ozone.

The following rationale was used to identify scavengers of superoxide anion which are effective in dry or near-dry compositions. Superoxide can act as both an oxidising free radical and reducing free radical. For example, it can reduce the haem Fe(III) in cytochrome c, and it can oxidise ascorbate ion. The oxidative power of superoxide increases in protonated form (HO₂.). However, due to a low pKa of superoxide in aqueous systems, the protonation is very unlikely and the reactivity of superoxide in aqueous solutions is considerably lower that that of other free radicals. Consequently, superoxide is believed not to contribute considerably to the radiation damage of proteins in aqueous solutions (Halliwell and Gutteridge, 1999). However, in low water activity systems (such as in organic solvents or in dry or near-dry systems) the ability of superoxide to accept protons is considerably increased and its oxidising ability therefore increases dramatically. In such systems, superoxide is known to act as an oxidising agent only towards compounds that can donate protons (Halliwell and Gutteridge, 1999). Since proteins contain multiple proton-donating sites and multiple oxidisable sites, the contribution of superoxide to the radiation damage in dry (or near-dry) systems increases considerably. So, as discussed above, in order to protect proteins against the effect of superoxide in dry state, it is necessary to add appropriate compounds capable of scavenging superoxide radical in dry state. Such compounds are those that meet both of the following two criteria:
they can be chemically oxidised (i.e. they are either strong or mild reducing agents)
they are capable of exchanging a proton readily with the superoxide radical (i.e. they contain a functional group capable of proton exchange with pKa no further than 3 pH units from the pH of the formulation, preferably no further than 2 pH units from the pH of the formulations and most preferably no further than 1 pH unit from the pH of the formulation).

Examples of such compounds comprise carboxylic acids (and salts thereof) containing one or more hydroxyl groups (e.g. lactic acid, citric acid, ascorbic acid, malic acid, tyrosine, thiamine etc.), carboxylic acids containing a thiol group (such as cysteine, thiosalicylic acid, thioglycolic acid etc.) and other compounds capable simultaneously of proton dissociation and chemical oxidation, such as histidine, methionine etc.

The rates of reaction of chemical species with the remaining oxygen radical species generated on radiolysis of oxygen in gaseous state (O₂⁺, O⁺ and O.) are not widely available in scientific sources. Similarly, it is difficult to find qualitative descriptions of the scavengers of these radical species. Therefore, a clear identification of effective “scavengers” of these free radicals is practically impossible for the purpose of the present invention, and the effects of O₂⁺, O⁺ and O. scavengers are thus of secondary importance in the present model. However, since all these radicals lack (and can thus be stabilised by) an electron, it can be assumed that compounds with low redox potential (i.e. reducing compounds that are likely to donate an electron) such as ascorbic acid, thiamine or the iodide anion, will act as scavengers of these species. However, the choice of the additive with low redox potential has to take into account the nature of the protein in question. In many cases, it is important to avoid strong reducing agents with very low redox potentials, such as ascorbic acid or cysteine, because such compounds can disrupt the disulphide bonds necessary to maintain the native structure of the protein. For example, human growth hormone is incompatible with ascorbic acid for this particular reason. Mild reducing agents (such as iodide or thiamine) are therefore generally preferable to strong reducing agents (such as ascorbate).

As a general rule of thumb, the following reasoning is suggested to distinguish between mild and strong oxidizing agent in the context of the present invention: the standard oxidation-reduction potential (E⁰) of the thiol/disulphide pair is generally between −0.2 V to −0.3 V. In those cases when it is important to prevent the reduction of disulphide bridge(s) in proteins, it is important to ensure that the added reducing agents have standard oxidation-reduction potentials significantly higher than −0.2 V. In contrast, adding reducing agents with E⁰ comparable or lower that that of the thiol/disulphide pair will generally result in reduction of the disulphide bridge(s). Consequently, an arbitrary measure was produced to distinguish between mild and strong oxidising agents as follows: “strong” reducing agents are those with E⁰<0.1 V; “mild” reducing agents are those with E⁰>0.1 V.

Examples of scavengers of the reactive oxygen species are shown in Table 1. The table lists only a limited number of potential scavengers of the selected reactive oxygen species and the present invention is by no means limited to the use of these compounds.

TABLE 1
Examples of scavengers of oxygen-derived reactive species. *Rate
constants were obtained from the Radiation Chemistry Data Center website.

| Oxygen radical | Quantitative data_*_Rate | |
|---|---|---|
| Scavengers | constant k (L mol⁻¹ s⁻¹) | Qualitative data |
| Singlet oxygen | | |
| Histidine | k = 4.6 × 10⁷ | Kawamoto et al. (1997) |
| Alanine | k = 3.0 × 10⁷ | |
| Tryptophan | k = 1.3 × 10⁷ | |
| Ascorbate anion | k = 1.5 × 10⁸ | |
| NADH | k = 2 × 10⁷ | |
| Thiourea | k = 6 × 10⁷ | |
| Thiamine | k = 6 × 10⁷ | |
| Cysteine | k = 5 × 10⁷ | |
| Cysteine anion | k = 1.5 × 10⁸ | |
| Azide anion | k = 4.5 × 10⁸ | |
| Copper(II) ion | k = 6.4 × 10⁷ | |
| Nickel (II) ion | k = 3.3 × 10⁷ | |
| Superoxide | | |
| Tiron | | Hardeland et al. (2003) |
| Ascorbate | | Sentman et al. (2006) |
| Mannitol | | Fornes et al. (1993) |
| Malate | | Purvis (2001) |
| Aminosalicylates (e.g. 5- | | Blazovics et al. (1999) |
| aminosalicylic acid) | | van den Berg et al. |
| Citrate | | (2003) |
| Superoxide scavengers effective | | (Halliwell and |
| in dry state | | Gutteridge, 1999). |
| Tiron | | |
| Citrate | | |
| Lactate | | |
| Malate | | |
| Tryptophan | | |
| Methionine | | |
| Cysteine | | |
| Thiosalicylate | | |
| Thioglycolate | | |
| Thiamine | | |
| Ozone | | |
| Pentoxifylline | | Keinan et al (2005) |
| Lisofylline | | Keinan et al (2005) |
| Limonene | | Keinan et al (2005) |
| Enprofylline | | Keinan et al (2005) |
| O₂⁺,O⁺ and O• | | |
| Strong oxidising agents | | |
| such as: | | |
| Ascorbic acid | | |
| Thiols (e.g. cysteine) | | |
| Mild oxidising agents such | | |
| as: | | |
| Thiamine | | |
| Iodide anion | | |

It was shown experimentally that each of the following types of compounds is capable of conferring a degree of protection to model proteins in the dry state through gamma irradiation:
Singlet oxygen scavengers
Superoxide scavengers (those effective in dry state, i.e. compounds capable simultaneously of proton dissociation and chemical oxidation)
Ozone scavengers
Mild reducing agents

However, none of the above types of compounds alone could confer stability of a model protein that would satisfy the requirements for therapeutic formulation. Such stability could only be achieved if the compounds were combined so that the composition contained at least one scavenger of singlet oxygen and at least one compound that is mild or strong reducing agent and that at the same time is capable of exchanging a proton readily with the superoxide radical (i.e. the compound contains a functional group capable of proton exchange with pKa no further than 3 pH units from the pH of the formulation, preferably no further that 2 pH units from the pH of the formulations and most preferably no further than 1 pH unit from the pH of the formulation). In the context of this invention, such a compound is referred to as “superoxide scavenger effective in dry state”.

Although ozone scavengers alone were capable of causing a degree of improvement of protein stability through ionising radiation, their importance was found limited in the combined formulations. This can be explained by the fact that ozone is a secondary product of oxygen radiolysis. So, the importance of ozone scavengers is limited, as long as the primary products are removed effectively by other additives. Nevertheless, ozone scavengers can still be used as optional excipients in combined formulations.

Similarly, a reducing agent (preferably a mild reducing agent with E⁰>0.1 V) that is not capable of exchanging protons with surrounding molecules can be optionally added to the formulation to improve further the stability of the formulation through ionising radiation.

Consequently, in order to achieve a degree of stability of dry protein through sterilisation by ionising radiation, the formulation should contain one of the following:
One or more singlet oxygen scavengers (i.e. a compound with the rate of reaction with singlet oxygen greater than 1×10⁷ L mol⁻¹ s⁻¹)
One or more superoxide scavengers effective in dry state
One or more ozone scavengers
One or more additional compounds with low redox potential (preferably a mild reducing agent with E⁰>0.1 V).

In order to achieve satisfactory stability of dry protein through sterilization by ionising radiation, the formulation should contain one of the following:
A combination of one or more singlet oxygen scavengers and one or more superoxide scavengers effective in dry state. Optionally, the formulation may contain an ozone scavenger.
A combination of one or more singlet oxygen scavengers and one or more compounds with low redox potential (preferably a mild reducing agent with E⁰>0.1. V). Optionally the formulation may contain an ozone scavenger.
A combination of one or more superoxide scavengers effective in dry state and one or more compounds with low redox potential (preferably a mild reducing agent with E⁰>0.1 V). Optionally, the formulation may contain an ozone scavenger.

In order to achieve the best stability of dry protein through sterilisation by ionising radiation that will satisfy the strict stability requirements for sterile therapeutic preparations, the formulation should contain one of the following:
A combination of one or more singlet oxygen scavengers, one or more superoxide scavengers effective in dry state and one or more compounds with low redox potential (preferably a mild reducing agent with E⁰>0.1 V). Optionally, the formulation may contain an ozone scavenger.

The required characteristics, namely the scavenging ability of singlet oxygen, superoxide (effective in dry state) and ozone, and the low redox potential may all be present in a single protective compound, but they are more likely to be separately present in two or more different compounds that together form a combination of protective compounds. It is also possible for several members of a combination of protective compounds to satisfy the same requirement.

The protection may be complete, i.e. with 100% retention of activity, so that no activity is lost on exposure to ionising radiation, or may be partial, with less than 100% retention of activity, so that some (but not all) activity is lost on exposure to ionising radiation. The retention of activity is preferably at least 50%, more preferably at least 60%, 70%, 80% or 90%, most preferably at least 95%.

The ionising radiation is typically in the form of gamma radiation, electron beam radiation or X-ray radiation.

The protective compound(s) may optionally be used in combination with other ingredients that may be desired or required in the protein formulations (e.g. antimicrobial agents, cofactors, bulking materials).

The pH of the formulation containing the protective compound(s) may be adjusted to a required value, for example a value that ensures best heat stability of the protein during and subsequent to the sterilisation. Typically, proteins will be formulated at pH between 4 to 9. Most therapeutic proteins or proteins used for diagnostic purposes will be formulated at pH 5 to 8, typically at pH 5 to 7, often around pH 6.

Small peptides comprising fewer than 20 amino acids, which contain at least one disulphide bridge, are likely to require formulating at pH between 4 to 6, typically around 5 to ensure optimum stability. This is because the stability of disulphide bond is best at pH between 4 to 5.

The term “protein” is used herein to encompass molecules or molecular complexes consisting of a single polypeptide, molecules or molecular complexes comprising two or more polypeptides and molecules or molecular complexes comprising one or more polypeptides together with one or more non-polypeptide moieties such as prosthetic groups, cofactors etc. The term “polypeptide” is intended to encompass polypeptides comprising covalently linked non-amino acid moieties such as glycosylated polypeptides, lipoproteins etc. In particular, the invention relates to molecules having one or more biological activities of interest, which activity or activities are critically dependent on retention of a particular or native three-dimensional structure in at least a critical portion of the molecule or molecular complex. In general it is thought the invention is applicable to polypeptides of any molecular weight. Examples of proteins are given in WO2007/003936, the content of which is incorporated herein for reference.

In general, especially with proteins for medical use, it will be desirable to use the compound(s) in as low a concentration as possible while still obtaining effective protection. The protective compound(s)/protein weight ratio is typically in the range 1-1000, preferably 5-200, most preferably 10-100.

The most preferred protein formulations, which comprise the single oxygen scavenger, scavenger of superoxide effective in dry state and optionally an additional mild reducing agent, and which thus provide the best stability of proteins, either for therapeutic or for diagnostic applications, during sterilization by ionising radiation, are listed in Table 2. The Table lists only a limited number of preferred mixtures of excipients and the present invention is not limited to the use of these formulations. The weight ratio between the excipients and the protein in these formulations is typically in the range 1-1000, preferably 5-200, and most preferably 10-100. The weight ratio between any two excipients in a formulation is typically in the range 1-10, preferably 1-5. The pH of the formulations can be adjusted to any required value, typically between 4 to 9. For most therapeutic proteins, the required pH range is typically between 5 to 7, often around 6. For small peptides (less than 20 amino acids) with a disulphide bridge, the optimum pH may however be lower, typically between 4 to 6, often around 5.

TABLE 2
Excipients present in the most preferred protein formulations
defined by the present invention

| Formulation No. | Excipients |
|---|---|
| 1 | Histidine + Citrate |
| 2 | Histidine + Tiron |
| 3 | Histidine + Lactate |
| 4 | Histidine + Methionine |
| 5 | Histidine + Malate |
| 6 | Histidine + Citrate + Iodide |
| 7 | Histidine + Tiron + Iodide |
| 8 | Histidine + Lactate + Iodide |
| 9 | Histidine + Methionine + Iodide |
| 10 | Histidine + Malate + Iodide |
| 11 | Thiamine + Citrate |
| 12 | Thiamine + Tiron |
| 13 | Thiamine + Lactate |
| 14 | Thiamine + Methionine |
| 15 | Thiamine + Malate |
| 16 | Thiamine + Citrate + Iodide |
| 17 | Thiamine + Tiron + Iodide |
| 18 | Thiamine + Lactate + Iodide |
| 19 | Thiamine + Methionine + Iodide |
| 20 | Thiamine + Malate + Iodide |
| 21 | Tryptophan + Citrate |
| 22 | Tryptophan + Tiron |
| 23 | Tryptophan + Lactate |
| 24 | Tryptophan + Methionine |
| 25 | Tryptophan + Malate |
| 26 | Tryptophan + Citrate + Iodide |
| 27 | Tryptophan + Tiron + Iodide |
| 28 | Tryptophan + Lactate + Iodide |
| 29 | Tryptophan + Methionine + Iodide |
| 30 | Tryptophan + Malate + Iodide |

The following Examples illustrate the invention. The Examples summarise the results of practical investigations into the protective effect of various potential protective compounds (singly or in combination) on the recovery of either measurable protein activity or measurable structural integrity after gamma sterilisation of dry formulations.

### Chemicals & Other Materials

Water (conductivity <10 μS cm⁻¹; either analytical reagent grade, Fisher or Sanyo Fistreem MultiPure)
Catalase (from bovine liver, Sigma C9322, 2380 U /mg solid)
Citric acid (Fisher, Code C/6200/53)
Deionised water (conductivity <10 μS cm⁻¹; either analytical reagent grade, Fisher or Sanyo Fistreem MultiPure)
Disodium hydrogen orthophosphate (Fisher, Code S/4520/53)
DMSO—Dimethyl sulfoxide (Sigma-Aldrich Code 154938-500)
Glucose (Fisher, Code G050061)
Glucose Oxidase (Biocatalysts G575P˜150 U/mg solid)
Human growth hormone standard was supplied by National Institute of Biological Standards and Control. Further samples for experimentation were obtained on prescription from a local GP surgery.
Hydrochloric acid (Fisher, Code J/4310/17)
Hydrogen peroxide (Sigma H1009)
Lactoperoxidase (from bovine milk, DMV International: 1,050 units mg−1 by ABTS method pH 5.0)
Potassium iodide (Fisher, Code 5880/53)
Sodium dihydrogen orthophosphate (Fisher, Code S/3760/60)
Starch (Acros Organics, Code 177132500)
TMB—Tetramethylbenzidine (Sigma T-2885)
Trizma base
n-Propyl alcohol

### Overall Experimental Plan

In each example, an aqueous solution of a protein was prepared with selected additives in an Eppendorf tube or in a glass vial. Water was removed from the formulation by drying under a stream of nitrogen at 30° C. and subsequent incubation at atmospheric pressure in the presence of a dessicant. The Eppendorf tubes or the glass vials were sealed and delivered to an industrial sterilisation service for gamma irradiation, with a dose range typical for sterile medical products. The gamma-irradiated samples were reconstituted on their return and analysed for protein activity or structural integrity. The results were compared with those achieved using control (i.e. non-irradiated) samples.

### Gamma Irradiation

The dry samples (approx. 20 μg in an Eppendorf tube) were gamma-irradiated by an industry-standard commercial sterilising service provided by Isotron PLC (Swindon, Wilts, UK), using a Cobalt 60 gamma source at ambient temperature. The radiation dose was in the range of 25-40 kGy.

### Glucose Oxidase Activity Assay

The original solutions (i.e. solutions prior to drying) contained 350 μg mL⁻¹ of glucose oxidase and typically the total of 100 mM of protective compounds (i.e. 100 mM in case of a single compound, 50 mM+50 mM in case of two compounds, 33.3 mM+33.3 mM+33.3 mM in the case of three compounds etc.). The solutions were dried and gamma irradiated. Following the gamma irradiation, the samples, both pre- and post-gamma irradiated, were assayed for glucose oxidase activity. This was performed according to the following procedure:

Water was added to the sample to achieve 350 μg mL⁻¹ of glucose oxidase. 50 μL of the solution was added to 50 mL of deionised water. The following solutions were then added:
10 mL of reagent mix (5 parts of 0.1 M sodium phosphate, pH 6+4 parts 2% w/w starch+1 part of 1 mg/mL lactoperoxidase enzyme);
5 mL of 100 mM potassium iodide; and
5 mL of 20% w/w glucose solution.

These were mixed together quickly. Time=0 was counted from the addition of the glucose. After 5 min, 1 ml of 5 M aq. hydrochloric acid was added to stop the reaction. The absorbance was then read at 630 nm using a Unicam UV-visible spectrophotometer (Type: Helios gamma). If the colour intensity was too great to allow an accurate reading, the sample was diluted with a defined volume of deionised water to bring the colour back on scale. The results were expressed as percentage recovery, by reference to the absorbance measured in the pre-gamma irradiation samples.

### Catalase Activity Assay

The original solutions (i.e. solutions prior to drying) contained 100 μg mL⁻¹ of catalase and typically the total of 100 mM of protective compounds (i.e. 100 mM in case of a single compound, 50 mM+50 mM in case of two compounds, 33.3 mM+33.3 mM+33.3 mM in the case of three compounds etc.). The solutions were dried and gamma irradiated. Following the gamma irradiation, the samples, both pre- and post-gamma irradiated, were assayed for glucose oxidase activity. This was performed according to the following procedure:

Water was added to the sample to achieve 100 μg mL⁻¹ of catalase. 100 μL of the solution was added to a mixture of 18 mL of PBS and 2 mL of hydrogen peroxide (30 mM in water) in a 125 mL polypropylene pot and mixed. The resulting mixture was incubated at room temperature precisely for 30 min. In the meantime, the following reagents were mixed in a plastic cuvette for spectrophotometric measurements:
2.73 mL of citrate/phosphate buffer (0.1 M, pH 5.0)
100 μL of tetramethylbenzidine (TMB) (3 mg/mL, dissolved in dimethyl sulphoxide (DMSO))
100 μL of lactoperoxidase

Following the 30 min incubation period, 70 μL of the catalase containing mixture was added to the cuvette and absorbance was read in approximately 30 s. The results were expressed as percentage recovery, by reference to the absorbance measured in the fresh samples (i.e. prior to incubation at increased temperature).

### Human Growth Hormone HPLC Assay

Mobile phase was prepared by mixing 71 parts (by volume) of a solution of TRIS (0.05 M, in water adjusted with hydrochloric acid to a pH of 7.5) and 29 parts (by volume) of n-propylalcohol. The mobile phase was filtered prior to its use. The liquid chromatograph (Agilent 1100 series) was equipped with a 214 nm detector and a 4.6×250 mm column (Phenomenex 00G-4167-E0) packed with butylsilyl silica gel with a granulometry of 5 μm and a porosity of 30 nm, maintained at 45° C. The flow rate was maintained at 0.5 mL min⁻¹. 15 μL of aqueous samples of human growth hormone (typically 1-2.5 mg mL⁻¹) were injected. Results were expressed as % of peak area corresponding to the gamma irradiated sample with respect to that measured in non-irradiated sample.

### Sandostatin HPLC Assay

Mobile phase A was 0.1 M triethylamine adjusted to pH 2.3 with phosphoric acid. Mobile phase B was acetonitrile. The mobile phases were filtered prior to their use. The following linear gradient was used: time 0: 90% A+10% B; time 35 min: 60% A+40% B. The liquid chromatograph (Agilent 1100 series) was equipped with a 214 nm detector, guard column and a 4.6×150 mm C18 column with a granulometry of 5 μm and a porosity of 30 nm, maintained at ambient temperature. The flow rate was maintained at 1.0 mL min⁻¹. Injection volume was 50 μL (typically sandostatin at 200 μg mL⁻¹). Results were expressed as % of main peak area (i.e. area of the peak corresponding to intact sandostatin measured in the gamma irradiated sample with respect to that measured in non-irradiated sample of identical composition). A chromatogram of a standard solution of sandostatin was recorded after every 12 samples to ensure that no drift in the position of the major peak had occurred. The control measurements ruled out any ambiguity in interpreting the chromatograms.

### EXAMPLE 1

### Effect of Selected Antioxidants on the Recovery of Activity of Model Proteins Following Gamma Irradiation

The effect of a selection of antioxidants suggested in US2003/0012687A1 was tested both on the recovery of functional activity of glucose oxidase and on the recovery of structural integrity of human growth hormone. Some of the antioxidants tested are known to be efficient scavengers of either singlet oxygen (ascorbate) or superoxide (ascorbate, urate, methionine).

The strong reducing ability of some of the compounds tested (namely ascorbate, cysteine and N-acetylcysteine) caused incompatibility with human growth hormone due to disruption of disulphide bridge. The capacity of these antioxidants to be used as excipients in therapeutic protein formulation is therefore very limited.

The antioxidants with weaker reducing ability were found compatible with the model proteins. Typically, the presence of these antioxidants improved the stability of the model proteins during sterilisation by ionising radiation at ambient temperature (see Table 3 and Table 4). In the case of glucose oxidase, the improved recovery was typically between 30-60%, the combination of ascorbate, urate and trolox resulting in the best recovery of 72.9%. In the case of human growth hormone, the best stability was achieved using methionine as sole excipient (69.7% recovery). Importantly, however, whilst a degree of stabilisation of proteins in dry state can be achieved using excipients disclosed in prior art such stability would not be sufficient to meet the criteria for stability of a therapeutic protein in a dry formulation during the sterilisation (i.e. >90%, but ideally >95% recovery). Achieving such recovery is addressed in the present invention.

TABLE 3
Activity recovery of glucose oxidase in dry formulations following
gamma irradiation. Concentration of glucose oxidase
in the original solution prior to drying was 350 μg mL⁻¹.

| | | Enzyme activity remaining |
|---|---|---|
| Compound | Concentration* | after gamma-irradiation |
| Control (i.e. original | — | 7.9% |
| formulation) | | |
| Ascorbate | 100 mM | 44.4% |
| Urate | 1.25 mM | 32.6% |
| Trolox | 0.75 mM | 45.9% |
| N-Acetylcysteine** | — | — |
| Cysteine | 100 mM | 49.1% |
| Methionine | 100 mM | 61.3% |
| Silymarine | 0.5 mM | 46.3% |
| Glycylglycine | 100 mM | 20.1% |
| Ascorbate + | 100 mM + | 30.2% |
| Glycylglycine | 100 mM | |
| Ascorbate + | 100 mM + | 59.7% |
| Urate | 1.25 mM | |
| Ascorbate + | 100 mM + | 72.9% |
| Urate + | 1.25 mM + | |
| Trolox | 0.75 mM | |
| Methionine + | 100 mM + | 65.0% |
| Glycylglycine | 100 mM | |
| Methionine + | 100 mM + | 64.8% |
| Trolox | 0.75 mM | |
| Ascorbate + | 100 mM + | 61.1% |
| Silymarine | 0.5 mM | |

*i.e. concentration in the original solution prior to drying
**Compound was found incompatible with glucose oxidase

TABLE 4
Recovery of structural integrity of human growth hormone
in dry formulations following gamma irradiation. Structural
integrity was assessed by HPLC. Concentration of human
growth hormone in the original solution prior to
drying was 2.5 mg mL⁻¹.

| | | Enzyme activity remaining |
|---|---|---|
| Compound | Concentration* | after gamma-irradiation |
| Control (i.e. original | — | 23.3% |
| formulation | | |
| Ascorbate** | — | — |
| Urate | 1.25 mM | 61.5% |
| Trolox | 0.75 mM | 49.6% |
| N-Acetylcysteine** | — | — |
| Cysteine** | — | — |
| Silymarine | 0.5 mM | 63.0% |
| Methionine | 100 mM | 69.7% |
| Glycylglycine | 100 mM | 47.2% |
| Urate + | 1.25 mM + | 51.1% |
| Trolox | 0.75 mM | |
| Methionine + | 100 mM + | 67.8% |
| Glycylglycine | 100 mM | |
| Methionine + | 100 mM + | 60.6% |
| Trolox | 0.75 mM | |
| Glycylglycine + | 100 mM + | 57.7% |
| Trolox | 0.75 mM | |

*i.e. concentration in the original solution prior to drying
**Compound was found incompatible with human growth hormone

### EXAMPLE 2

### Effect of a Selection of Singlet Oxygen Scavengers on the Recovery of Activity of Model Proteins Following Gamma Irradiation

The presence of selected singlet oxygen scavengers in the dry formulations of glucose oxidase (Table 5), catalase (Table 6), human growth hormone (Table 7) and Sandostatin (Table 8) improved the activity recovery (glucose oxidse, catalase) or structural recovery (human growth hormone, Sandostatin) following gamma irradiation. The recovery of the proteins following gamma irradiation in the absence of singlet oxygen scavengers varied considerably depending on the protein. The magnitude of the stabilising effect of singlet oxygen scavengers also varied depending both on the protein and on the particular excipient. Importantly, however, in no case was the stabilizing effect sufficient to meet the requirements for protein stability during sterilization of therapeutic formulations by ionising radiation. Ascorbate was found compatible with glucose oxidase and catalase and could therefore be tested as an excipient. In contrast, incorporation of ascorbate both in human growth hormone formulation and in Sandostatin formulation led to reduction of the disulphide bonds and subsequent degradation as detected by HPLC.

TABLE 5
Activity recovery of glucose oxidase in dry formulations
following gamma irradiation. Concentration of glucose oxidase
in the original solution prior to drying was 350 μg mL⁻¹.

| | | Rate of reaction | Enzyme activity |
|---|---|---|---|
| | | with singlet | remaining after |
| | | oxygen | gamma- |
| Compound | Concentration* | (L mol⁻¹ s⁻¹) | irradiation |
| Control (i.e. | — | — | 7.9% |
| original | | | |
| formulation) | | | |
| Histidine | 100 mM | 4.6 × 10⁷ | 57.9% |
| Methionine | 100 mM | 1.3 × 10⁷ | 61.3% |
| Alanine | 100 mM | 3.0 × 10⁷ | 15.3% |
| Tryptophan | 30 mM | 1.3 × 10⁷ | 37.0% |
| Thiamine | 50 mM | 6.0 × 10⁷ | 43.1% |

*i.e. concentration in the original solution prior to drying.

TABLE 6
Activity recovery of catalase in dry formulations following
gamma irradiation. Concentration of catalase in the original
solution prior to drying was 100 μg mL⁻¹.

| | | Rate of reaction | Enzyme activity |
|---|---|---|---|
| | | with singlet | remaining after |
| | | oxygen | gamma- |
| Compound | Concentration* | (L mol⁻¹ s⁻¹) | irradiation |
| Control (i.e. | — | — | 5.0% |
| original | | | |
| formulation) | | | |
| Histidine | 100 mM | 4.6 × 10⁷ | 18.3% |
| Alanine | 100 mM | 3.0 × 10⁷ | 8.8% |
| Tryptophan | 30 mM | 1.3 × 10⁷ | 9.1% |
| Thiamine | 50 mM | 6.0 × 10⁷ | 26.1% |

*i.e. concentration in the original solution prior to drying

TABLE 7
Recovery of structural integrity of human growth hormone in dry
formulations following gamma irradiation. Structural integrity was
assessed by HPLC. Concentration of human growth hormone in
the original solution prior to drying was 2.5 mg mL⁻¹.

| | | Rate of reaction | Enzyme activity |
|---|---|---|---|
| | | with singlet | remaining after |
| | | oxygen | gamma- |
| Compound | Concentration* | (L mol⁻¹ s⁻¹) | irradiation |
| Control (i.e. | — | — | 23.3% |
| original | | | |
| formulation) | | | |
| Histidine | 50 mM | 4.6 × 10⁷ | 55.5% |
| Methionine | 100 mM | 1.3 × 10⁷ | 69.7% |
| Tryptophan | 30 mM | 1.3 × 10⁷ | 54.6% |
| Thiamine | 50 mM | 6.0 × 10⁷ | 48.8% |

*i.e. concentration in the original solution prior to drying.

TABLE 8
Recovery of structural integrity of Sandostatin in dry formulations
following gamma irradiation. Structural integrity was assessed
by HPLC.

| | | Rate of | Enzyme activity |
|---|---|---|---|
| | | reaction with | remaining after |
| | Excipient:active | singlet oxygen | gamma- |
| Compound | ratio* | (L mol⁻¹ s⁻¹) | irradiation |
| Control (i.e. | — | — | 78.8% |
| original | | | |
| formulation) | | | |
| Histidine | 10:1 | 4.6 × 10⁷ | 85.0% |
| Methionine | 10:1 | 1.3 × 10⁷ | 91.1% |
| Tryptophan | 10:1 | 1.3 × 10⁷ | 82.2% |
| Thiamine | 10:1 | 6.0 × 10⁷ | 87.7% |

*i.e. weight ratio between the excipient and Sandostatin in the formulation.

### EXAMPLE 3

### Effect of a Selection of Superoxide Scavengers on the Recovery of Activity of Model Proteins Following Gamma Irradiation

Effect of superoxide scavengers was investigated on the stability of selected proteins during sterilisation by gamma radiation. With one exception, the superoxide scavengers tested were effective in dry state, i.e. they were capable of exchanging a proton with superoxide anion. The one exception was mannitol. The presence of superoxide scavengers in the dry formulations of glucose oxidase (Table 9), catalase (Table 10), human growth hormone (Table 11) and Sandostatin (12) improved the activity recovery (glucose oxidase, catalase) or structural recovery (human growth hormone, Sandostatin) following gamma irradiation. The magnitude of the effect varied depending both on the protein and on the excipient. In most cases, the effect of mannitol was considerably smaller compared with the effects of superoxide scavengers effective in dry state. It was only in the case of catalase that the effect of mannitol was comparable with that of the scavengers effective in dry state. This is very likely due to improvement of heat stability of the very labile catalase by mannitol. Importantly, however, in no case was the stabilising effect of any of the superoxide scavengers sufficient to meet the requirements for protein stability during sterilisation of therapeutic formulations by ionising radiation.

TABLE 9
Activity recovery of glucose oxidase in dry formulations
following gamma irradiation. Concentration of glucose oxidase
in the original solution prior to drying was 350 μg mL⁻¹.

| | | Enzyme activity remaining |
|---|---|---|
| Compound | Concentration* | after gamma-irradiation |
| Control | — | 7.9% |
| (i.e. original formulation | | |
| Mannitol | 200 mM | 17.2% |
| Tiron | 100 mM | 27.5% |
| Malate | 100 mM | 40.7% |
| Citrate | 100 mM | 32.0% |
| Methionine | 100 mM | 61.3% |

*i.e. concentration in the original solution prior to drying.

TABLE 10
Activity recovery of catalase in dry formulations following
gamma irradiation. Concentration of catalase in the original
solution prior to drying was 100 μg mL⁻¹.

| | | Enzyme activity remaining |
|---|---|---|
| Compound | Concentration* | after gamma-irradiation |
| Control | — | 7.9% |
| (i.e. original formulation | | |
| Mannitol | 200 mM | 36.3% |
| Tiron | 100 mM | 17.9% |
| Malate | 100 mM | 40.9% |
| Citrate | 100 mM | 39.5% |
| Methionine | 100 mM | 44.3% |

*i.e. concentration in the original solution prior to drying.

TABLE 11
Recovery of structural integrity of human growth hormone in dry
formulations following gamma irradiation. Structural integrity was
assessed by HPLC. Concentration of human growth hormone in
the original solution prior to drying was 2.5 mg mL⁻¹.

| | | Enzyme activity remaining |
|---|---|---|
| Compound | Concentration* | after gamma-irradiation |
| Control | — | 23.3% |
| (i.e. original formulation | | |
| Mannitol | 200 mM | 28.3% |
| Tiron | 125 mM | 55.7% |
| Malate | 125 mM | 52.1% |
| Citrate | 125 mM | 45.6% |
| Methionine | 100 mM | 69.7% |

*i.e. concentration in the original solution prior to drying

TABLE 12
Recovery of structural integrity of Sandostatin in dry formulations
following gamma irradiation. Structural integrity was assessed
by HPLC.

| | Excipient:active | Enzyme activity remaining |
|---|---|---|
| Compound | ratio* | after gamma-irradiation |
| Control | — | 78.8% |
| (i.e. original formulation | | |
| Mannitol | 10:1 | 72.6% |
| Tiron | 10:1 | 89.7% |
| Malate | 10:1 | 87.7% |
| Citrate | 10:1 | 91.8% |
| Methionine | 10:1 | 91.1% |

*i.e. weight ratio between the excipient and Sandostatin in the formulation.

### EXAMPLE 4

### Effect of a Selection of Ozone Scavengers on the Recovery of Activity of Model Proteins Following Gamma Irradiation

Effect of two ozone scavengers was tested on the recovery of the activity of model proteins following gamma irradiation. Both scavengers improved the recovery of glucose oxidase (Table 13). Eucaliptol was found to inhibit catalase, so its effect on recovery through gamma irradiation could not be tested. Nevertheless, pentoxyfylline, the other ozone scavenger tested improved the catalase recovery considerably (Table 14). Similarly, some improvement of structural integrity of human growth hormone on exposure to ionising radiation was observed in the presence of pentoxyfilline (Table 15). Importantly, however, in no case was the stabilising effect sufficient to meet the requirements for protein stability during sterilisation of therapeutic formulations by ionizing radiation.

TABLE 13
Activity recovery of glucose oxidase in dry formulations
following gamma irradiation. Concentration of glucose oxidase
in the original solution prior to drying was 350 μg mL⁻¹.

| | | Enzyme activity remaining |
|---|---|---|
| Compound | Concentration* | after gamma-irradiation |
| Control | — | 7.9% |
| (i.e. original formulation) | | |
| Pentoxyfylline | 100 mM | 36.2% |
| Eucaliptol | Pure** | 17.5% |

*i.e. concentration in the original solution prior to drying
**i.e. enzyme was dissolved directly in the protecting compound.

TABLE 14
Activity recovery of catalase in dry formulations following
gamma irradiation. Concentration of catalase in the original
solution prior to drying was 100 μg mL⁻¹.

| | | Enzyme activity remaining |
|---|---|---|
| Compound | Concentration* | after gamma-irradiation |
| Control | — | 5% |
| (i.e. original formulation) | | |
| Pentoxyfylline | 100 mM | 77.0% |

*i.e. concentration in the original solution prior to drying

TABLE 15
Recovery of structural integrity of human growth hormone in dry
formulations following gamma irradiation. Structural integrity was
assessed by HPLC. Concentration of human growth hormone in
the original solution prior to drying was 2.5 mg mL⁻¹.

| | | Enzyme activity remaining |
|---|---|---|
| Compound | Concentration* | after gamma-irradiation |
| Control | — | 23.3% |
| (i.e. original formulation) | | |
| Pentoxyfylline | 80 mM | 46.0% |

*i.e. concentration in the original solution prior to drying

### EXAMPLE 5

### Effect of Selected Combinations of Singlet Oxygen Scavengers, Scavengers of Superoxide, and Other Reducing Species on the Recovery of Activity of Model Proteins Following Gamma Irradiation

In general, the presence of various combinations of scavengers of singlet oxygen, scavengers of superoxide and reducing agents conferred better protection of glucose oxidase (Table 16), catalase (Table 17), human growth hormone (Table 18) and Sandostatin (Table 19) in dry formulations compared with the effect of single compounds (Examples 2, 3 and 4). However, in order to achieve the best stability of the proteins it was essential to include at least one singlet oxygen scavenger and at least one scavenger of superoxide effective in dry state. The presence of an additional reducing agent (preferably mild reducing agent) improved the stability even further in some cases. Only such formulations resulted in sufficient stability of the protein during sterilisation by gamma radiation to be considered for either therapeutic or diagnostic use.

TABLE 16
Activity recovery of glucose oxidase in dry formulations
following gamma irradiation. Concentration of glucose oxidase in
the original solution prior to drying was 350 μg mL⁻¹.

| | | | Enzyme |
|---|---|---|---|
| | | | activity |
| | | | remaining |
| | | | after |
| Combination of | | | gamma- |
| excipients | Concentration* | Excipient effect | irradiation |
| Control | — | — | 7.9% |
| (i.e. original | | | |
| formulation) | | | |
| Tiron + | 50 mM + | Superoxide scavenger | 56.6% |
| Malate | 50 mM | Superoxide scavenger | |
| Tiron + | 50 mM + | Superoxide scavenger | 62.1% |
| Ascorbate | 50 mM | Superoxide scavenger & | |
| | | Strong reducing agent | |
| Tiron + | 33.3 mM + | Superoxide scavenger | 55.1% |
| Malate + | 33.3 mM + | Superoxide scavenger | |
| Ascorbate | 33.3 mM | Superoxide scavenger & | |
| | | Reducing agent | |
| Tiron + | 33.3 mM + | Superoxide scavenger | 59.2% |
| Mannitol + | 33.3 mM + | Superoxide scavenger | |
| Thiamine | 33.3 mM | Reducing agent | |
| Tiron + | 50 mM + | Superoxide scavenger | 57.8% |
| Histidine | 50 mM | Singlet oxygen | |
| | | scavenger | |
| Tiron + | 50 mM + | Superoxide scavenger | 52.9% |
| Tryptophan | 50 mM | Singlet oxygen | |
| | | scavenger | |
| Tiron + | 33.3 mM + | Superoxide scavenger | 63.3% |
| Histidine + | 33.3 mM + | Singlet oxygen | |
| Mannitol | 33.3 mM | scavenger | |
| | | Superoxide scavenger | |
| Malate + | 33.3 mM + | Superoxide scavenger | 94.7% |
| Histidine + | 33.3 mM + | Singlet oxygen | |
| | | scavenger | |
| Citrate + | 33.3 mM + | Superoxide scavenger | 84.7% |
| Methionine + | 33.3 mM + | Superoxide scavenger | |
| Histidine + | 33.3 mM | Singlet oxygen | |
| | | scavenger | |
| Citrate + | 33.3 mM + | Superoxide scavenger | 94.1% |
| Histidine + | 33.3 mM + | Singlet oxygen | |
| KI | 33.3 mM | scavenger | |
| | | Reducing agent | |
| Tiron + | 50 mM + | Superoxide scavenger | 86.1% |
| Thiamine | 50 mM | Singlet oxygen | |
| | | scavenger | |
| | | Reducing agent | |
| Tiron + | 33.3 mM + | Superoxide scavenger | 95.0% |
| Histidine + | 33.3 mM + | Singlet oxygen | |
| Ascorbate | 33.3 mM | scavenger | |
| | | Superoxide scavenger & | |
| | | Reducing agent | |
| Methionine + | 50 mM + | Superoxide scavenger | 96.1% |
| Histidine | 50 mM | Singlet oxygen | |
| | | scavenger | |

*i.e. concentration in the anginal solution prior to drying.

TABLE 17
Activity recovery of catalase in dry formulations following
gamma irradiation. Concentration of catalase in the original
solution prior to drying was 100 μg mL⁻¹.

| | | | Enzyme |
|---|---|---|---|
| | | | activity |
| | | | remaining |
| | | | after |
| Combination of | | | gamma- |
| excipients | Concentration* | Excipient effect | irradiation |
| Control (i.e. | — | — | 5.0% |
| original | | | |
| formulation) | | | |
| Mannitol + | 50 mM + | Superoxide scavenger | 34.8% |
| Histidine | 50 mM | Singlet oxygen scavenger | |
| Tiron + | 50 mM + | Superoxide scavenger | 34.7% |
| Histidine | 50 mM | Singlet oxygen scavenger | |
| Mannitol + | 33.3 mM + | Superoxide scavenger | 79.5% |
| Histidine + | 33.3 mM + | Singlet oxygen scavenger | |
| Ascorbate | 33.3 mM | Singlet oxygen scavenger & | |
| | | Reducing agent | |
| Malate + | 50 mM + | Superoxide scavenger | 78.2% |
| Histidine | 50 mM | Singlet oxygen scavenger | |
| Malate + | 33.3 mM + | Superoxide scavenger | 78.3% |
| Histidine + | 33.3 mM + | Singlet oxygen scavenger | |
| Mannitol | 33.3 mM | Superoxide scavenger | |
| Histidine + | 50 mM + | Singlet oxygen scavenger | 29.8% |
| Tryptophan | 50 mM | Singlet oxygen scavenger | |
| Histidine + | 33.3 mM + | Singlet oxygen scavenger | 35.9% |
| Tryptophan + | 33.3 mM + | Singlet oxygen scavenger | |
| Mannitol | 33.3 mM | Superoxide scavenger | |
| Histidine + | 25 mM + | Singlet oxygen scavenger | 41.8% |
| Tryptophan + | 25 mM + | Singlet oxygen scavenger | |
| Mannitol + | 25 mM + | Superoxide scavenger | |
| Tiron | 25 mM | Superoxide scavenger | |
| Histidine + | 33.3 mM + | Singlet oxygen scavenger | 55.9% |
| Mannitol + | 33.3 mM + | Superoxide scavenger | |
| Tiron | 33.3 mM | Superoxide scavenger | |
| Malate + | 50 mM + | Superoxide scavenger | 55.8% |
| Ascorbate | 50 mM | Singlet oxygen scavenger & | |
| | | Reducing agent | |
| Malate + | 33.3 mM + | Superoxide scavenger | 98.7% |
| Histidine + | 33.3 mM + | Singlet oxygen scavenger | |
| Ascorbate | 33.3 mM | Singlet oxygen scavenger & | |
| | | Reducing agent | |

*i.e. concentration in the original solution prior to drying.

TABLE 18
Recovery of structural integrity of human growth hormone in dry
formulations following gamma irradiation. Structural integrity was
assessed by HPLC. Concentration of human growth hormone in
the original solution prior to drying was 2.5 mg mL⁻¹.

| | | | Enzyme |
|---|---|---|---|
| | | | activity |
| | | | remaining |
| | | | after |
| Combination of | Concen- | | gamma- |
| excipients | tration* | Excipient effect | irradiation |
| Control | — | — | 23.3% |
| (i.e. original | | | |
| formulation) | | | |
| Histidine + Iodide | 50 mM + | Singlet oxygen scavenger | 81.2% |
| | 80 mM | Reducing agent | |
| Tryptophane + | 20 mM + | Singlet oxygen scavenger | 66.2% |
| Iodide | 80 mM | Reducing agent | |
| Histidine + Citrate | 50 mM + | Singlet oxygen scavenger | 88.2% |
| | 50 mM | Superoxide scavenger | |
| Histidine + Citrate + | 50 mM + | Singlet oxygen scavenger | 94.1% |
| Iodide | 50 mM + | Superoxide scavenger | |
| | 80 mM | Reducing agent | |
| Tiron + Thiamine | 80 mM + | Singlet oxygen scavenger | 95% |
| | 80 mM | Superoxide scavenger | |
| | | Reducing agent | |
| Tiron + Thiamine + | 80 mM + | Superoxide scavenger | 98.3% |
| Histidine | 80 mM + | Reducing agent | |
| | 50 mM | Singlet oxygen scavenger | |
| Histidine + | 50 mM + | Singlet oxygen scavenger | 92.9% |
| Methionine | 50 mM | Superoxide scavenger | |
| Tryptophan + | 20 mM + | Singlet oxygen scavenger | 91.8% |
| Methionine | 50 mM | Superoxide scavenger | |

*i.e. concentration in the original solution prior to drying

TABLE 19
Recovery of structural integrity of Sandostatin in dry formulations
following gamma irradiation. Structural integrity was assessed
by HPLC.

| | | | Enzyme |
|---|---|---|---|
| | | | activity |
| | | | remaining |
| | | | after |
| Combination of | Excipient:active | | gamma- |
| excipients | ratio* | Excipient effect | irradiation |
| Control (i.e. | | — | 78.8% |
| original | | | |
| formulation) | | | |
| Histidine + | | Singlet oxygen scavenger | 95.9% |
| Citrate | | Superoxide scavenger | |
| Histidine + | | Singlet oxygen scavenger | 98.8% |
| Citrate + | | Superoxide scavenger | |
| Iodide | | Reducing agent | |
| Histidine + | | Singlet oxygen scavenger | 93.3% |
| Malate | | Superoxide scavenger | |
| Histidine + | | Singlet oxygen scavenger | 95.6% |
| Malate + | | Superoxide scavenger | |
| KI | | Reducing agent | |
| Thiamine + | | Singlet oxygen scavenger | 95.8% |
| Tiron | | Superoxide scavenger | |

*i.e. weight ratio between the excipient and Sandostatin in the formulation.

### REFERENCES

Blazovics et al (1999) Clinical Chemistry 45, 895-896.

Fornes et al (1993) Biochem. Biophys. Res. Commun. 195(3), 1289-1293.

Halliwell B. (1995) Free Rad. Biol. Med. 18, 125-126.

Halliwell B. and Gutteridge J. M. C (1999) Free radicals in Biology and Medicine. Third edition. Oxford University Press, ISBN 1-29-850044-0/45-0.

Hardeland et al (2003) J. Pineal Res. 34(1), 17-25.

Kawamoto et al (1997) No To Shinkei 49(7), 612-618.

Keinan et al (2005) Bioorg. Med. Chem. 13, 557-562.

Purvis A. C. (2001) J. Plant Physiol. 158(2) 159-165.

Sentman et al (2006) J. Biol. Chem. 281 (11):6904-6909.

Van den Berg et al (2003) J. Wound Care 12(10) 413-418.

While this invention has been particularly shown and described with references to preferred embodiments thereof, it will be understood by those skilled in the art that various changes in form and details may be made therein without departing from the scope of the invention encompassed by the appended claims.

1. A method of sterilising a protein, comprising exposing to ionising radiation an at least substantially dry composition comprising a protein and a protective compound or combination of protective compounds having both of the following characteristics:
(i) a rate of reaction with singlet oxygen greater than 1×10⁷ L mol⁻¹ s⁻¹; and
(ii) being a reducing agent whilst at the same time containing a proton dissociable group with a pKa no more than 3 units from the pH of the composition.

2. A method according to claim 1, wherein the reducing agent is a mild reducing agent with E⁰>+0.1 V.

3. A method according to claim 1, wherein the composition further comprises an additional reducing agent which is not capable of proton dissociation.

4. A method according to claim 3, wherein the additional reducing agent is a mild reducing agent with E⁰>+0.1 V.

5. A method according to claim 1, wherein the composition further comprises a scavenger of ozone.

6. A method according to claim 1, wherein the protective compound(s):protein weight ratio is in the range 1-100:1.

7. A method according to claim 1, wherein the pH of the composition is 4 to 9.

8. A method according to any preceding claim 1, wherein a compound having characteristic (i) is selected from histidine, thiamine and tryptophan.

9. A method according to claim 8, wherein a compound having characteristic (ii) is selected from methionine, malate, citrate, lactate and tiron.

10. A method according to claim 1, wherein the composition additionally comprises one or more additives selected from antimicrobial agents, cofactors, surfactants and bulking materials.

11. A method according to claim 1, which is conducted at ambient temperature.

12. A method according to claim 1, wherein the ionizing radiation is gamma radiation or electron beam radiation at a dose of 15-40 kGy.

13. (canceled)

14. A method according to claim 1, wherein the protein retains at least 80% activity on irradiation.

15. A method according to claim 1, wherein the protein retains at least 95% activity on irradiation.

16. A method according to claim 1, wherein the composition is physiologically acceptable.

17. A method according to claim 1, wherein the water content of the composition is no more than 5% by weight.

18. A method according to claim 1, wherein the composition comprises a combination of protective compounds having characteristics (i) and (ii), respectively.

19. A substantially dry sterile composition comprising a protein and a protective compound or combination as defined in claim 18 wherein the water content is no more than 5% by weight.

20. A composition according to claim 19, which is sterile, for therapeutic use or diagnostic use.

21-23. (canceled)
